(19) United States
(12) Patent Application Publication
(10) Pub. No.: US 2022/0017887 A1
(43) Pub. Date: Jan. 20, 2022

(54) COMPOSITIONS, KITS AND METHODS USEFUL FOR SEPARATING OLIGONUCLEOTIDES FROM MATRIX COMPONENTS

(71) Applicant: Waters Technologies Corporation, Milford, MA (US)

(72) Inventors: Michael Donegan, Charlton, MA (US); Martin Gilar, Franklin, MA (US); Darryl W. Brousmiche, Grafton, MA (US)

(21) Appl. No.: 17/375,325

(22) Filed: Jul. 14, 2021

Related U.S. Application Data
(60) Provisional application No. 63/180,878, filed on Apr. 28, 2021.
(60) Provisional application No. 63/051,595, filed on Jul. 14, 2020.

(51) Int. Cl. C12N 15/10; B01J 41/10; B01J 41/09

(52) U.S. Cl. CPC C12N 15/101; B01J 41/09; B01J 41/10

(57) ABSTRACT

The present disclosure relates to compositions, kits and methods that may be used for removal of matrix components, including proteins and lipids, from one or more oligonucleotides.

### CROSS-REFERENCE TO RELATED APPLICATIONS

“This application claims the benefit of U.S. Provisional Application No. 63/051,595, filed Jul. 14, 2020 and U.S. Provisional Application No. 63/180,878, filed Apr. 28, 2021, entitled “COMPOSITIONS, KITS AND METHODS USEFUL FOR SEPARATING OLIGONUCLEOTIDES FROM MATRIX COMPONENTS”, the entire disclosures of which are hereby incorporated by reference.

### FIELD

The present disclosure relates to compositions, kits and methods that may be used for removal of matrix components, including proteins and lipids, from oligonucleotides.

### BACKGROUND

Oligonucleotides are polymeric sequences of nucleotides (RNA, DNA, their analogs, and their derivatives) that are utilized extensively as PCR (polymerase chain reaction) and microarray-based reagents in life science research and oligonucleotide-based diagnostic test kits (as primer and probe reagents). Oligonucleotides are also being developed as therapeutic drugs for a wide range of disease conditions. Oligonucleotides developed as therapeutics can take a variety of forms, including antisense oligonucleotides (ASOs), small interfering RNAs (siRNAs), small hairpin RNAs (shRNAs), and micro RNAs (miRNAs) that can effect “gene silencing,” which is the downregulating or turning off the expression of specific genes/proteins, aptamers that behave like small molecule drugs and bind to specific disease targets, and messenger RNAs (mRNAs) that can be very long, and are being designed to up-regulate expression of particular proteins, and plasmids (duplex DNA).

Extraction of oligonucleotides from complex biological matrices such as plasma, blood, urine and tissue present a difficult analytical challenge. The polyanionic nature of oligonucleotides ensure that these compounds will be strongly bound to plasma proteins in addition to other matrix components. Successful bioanalytical sample preparation hinges on the difficult process of separating the oligonucleotide from the matrix. The most common approach to extraction involves a multistep liquid-liquid extraction followed by an additional solid phase extraction (SPE) to further clean up the sample. This approach is popular because it will invariably successfully extract oligonucleotides. However, multiple steps are involved in the process, which introduces a source of error in reproducibility and increases time required, making this method inefficient.

With an explosion of oligonucleotide research programs being conducted in industry, the development of a fast, universal SPE solution for the bioanalysis of oligonucleotides is desired.

### SUMMARY

In accordance with some aspects, the present disclosure pertains to sorbents that comprise a bulk material and ionizable surface groups having a pKa in a range of about 8 to about 12, more typically about 9 to about 12, even more typically about 10 to about 12.

In accordance with some aspects, the present disclosure pertains to methods of performing solid phase extraction comprising: (a) loading a sample comprising one or more target oligonucleotides and one or more matrix components comprising proteins, lipids or both, onto a porous anion exchange sorbent comprising a bulk material and ionizable surface groups having a pKa in a range of about 8 to about 12, more typically about 9 to about 12, even more typically about 10 to about 12, wherein target oligonucleotides are retained by the sorbent and matrix components are retained or unretained by the sorbent (in various embodiments, at least a portion of the matrix components are retained by the sorbent); (b) flowing one or more washing solutions through the sorbent, wherein the washing solutions remove retained matrix components from the sorbent while leaving the target oligonucleotides retained on the sorbent; and (c) flowing one or more elution solutions though the sorbent, wherein the target oligonucleotides retained on the sorbent are released.

In various embodiments, the ionizable surface groups may comprise amine-containing groups. For example, the amine-containing groups may be selected from —NHR₁ groups, —NR₁R₂ groups, and heterocyclic ring systems that contain at least one nitrogen atom, where R₁ and R₂ are independently selected from C₁-C₁₈ alkyl, C₂-C₁₈ alkenyl, C₂-C₁₈ alkynyl, C₃-Cis cycloalkyl, C₃-C₁₈ heterocycloalkyl, C₆-C₁₈ aryl, or C₅-C₁₈ heteroaryl. In particular embodiments, the amine-containing groups comprise diethylaminopropyl (DEAP), ethylaminopropyl, dimethylaminopropyl, methylaminopropyl, aminopropyl, or diethylaminomethyl groups.

In various embodiments, which can be used with any of the above aspects and embodiments, the amine-containing groups are linked to the bulk material by linking moieties. For example, the linking moieties may comprise one or more of alkyl groups, amide groups, ester groups, sulfo groups, ether groups, carbamate groups and urea groups. In certain embodiments, the linking moieties may comprise an amide group, ester group, sulfo group, ether group, carbamate group or urea group positioned between two C₁-C₆-alkyl groups.

In various embodiments, which can be used with any of the above aspects and embodiments, the bulk material comprises an inorganic material, a inorganic-organic hybrid material, an organic polymeric material, or a combination thereof.

In various embodiments, which can be used with any of the above aspects and embodiments, the bulk material may comprise a silica-based material.

In various embodiments, which can be used with any of the above aspects and embodiments, the bulk material may comprise an inorganic-organic hybrid material that comprises silica regions in which the material comprises silicon atoms having four silicon-oxygen bonds and organosilica regions in which the material comprises silicon atoms having one or more silicon-oxygen bond and one or more silicon-carbon bonds. The organosilica regions may comprise, for example, a substituted or unsubstituted alkylene, alkenylene, alkynylene or arylene moiety bridging two or more silicon atoms.

In various embodiments, which can be used with any of the above aspects and embodiments, the bulk material may comprise silanol groups at a surface of a silica-based material, which are reduced in concentration by reaction with a C₁-C₁₈ alkyl silane compound.

In various embodiments, which can be used with any of the above aspects and embodiments, the porous anion exchange sorbent employed may be in monolithic form or in particulate form.

In various embodiments, which can be used with any of the above aspects and embodiments, the one or more target oligonucleotides have a size ranging from a 3 mer to a 7000 mer.

In various embodiments, which can be used with any of the above aspects and embodiments, (a) the porous anion exchange sorbent has a pore size ranging from 75 to 200 Angstroms and the sample contains one or more target oligonucleotides having a size ranging a 3 mer to a 50 mer, (b) the porous anion exchange sorbent has a pore size ranging from 200 to 500 Angstroms and the sample contains one or more target oligonucleotides having a size ranging a 25 mer to a 200 mer, and/or (c) the porous anion exchange sorbent has a pore size ranging from 500 to 2000 Angstroms and the sample contains one or more target oligonucleotides having a size ranging a 100 mer to a 7000 mer.

In various embodiments, which can be used with any of the above aspects and embodiments, the one or more washing solutions may comprise an organic solvent and a volatile buffer.

In various embodiments, which can be used with any of the above aspects and embodiments, the one or more elution solutions may have a pH ranging from 10 to 13.

In various embodiments, which can be used with any of the above aspects and embodiments, the one or more elution solutions may comprise a polyphosphonic acid.

In various embodiments, which can be used with any of the above aspects and embodiments, the one or more elution solutions may comprise one or more bases selected from an organic amine, ammonium bicarbonate, ammonium hydroxide, or ammonium acetate and one or more organic solvents selected from methanol, ethanol, hexafluoroisopropanol or tetrahydrofuran. In certain embodiments, the one or more elution solutions may comprise triethylamine (TEA), methanol and water, or the one or more elution solutions may comprise TEA, methanol, hexafluoroisopropanol and water.

In various embodiments, which can be used with any of the above aspects and embodiments, the sample comprises biological fluids selected from whole blood samples, blood plasma samples, serum samples, oral fluids, cerebrospinal fluids, fecal samples, nasal samples, and urine, biological tissues such as liver, kidney and brain tissue, tissue homogenates, cells, or cell culture supernatants.

In various embodiments, which can be used with any of the above aspects and embodiments, the method further comprises treating the sample with a denaturing agent before loading the sample onto the porous anion exchange sorbent. For example, the denaturing agent may be selected from a protease such as proteinase K, an MS compatible surfactant, an organic solvent, urea, guanidine, or a substituted guanidine.

In various embodiments, which can be used with any of the above aspects and embodiments, the present disclosure pertains to kits that comprise a porous anion exchange sorbent comprising ionizable surface groups having a pKa in a range of about 8 to about 12 (for example, a porous anion exchange sorbent in accordance with any of the preceding aspects and embodiments), a housing for the sorbent, and one or more kit components selected from the following: a denaturant solution, an elution solution, or a washing solution, among others.

In some embodiments, the housing may be selected from a multi-well strip, a multi-well plate, a single-use cartridge, or a multiple-use cartridge configured for on-line SPE.

These and other aspects and embodiments of the disclosure will become apparent upon review of the Detailed Description to follow.

### BRIEF DESCRIPTION OF THE DRAWINGS

FIG. 1 is a calibration curve for GEM 91 showing linear recovery using an Oasis™_based 20 μm 500 Å-DEA SPE plate.

FIG. 2 is a calibration curve for GEM 91 showing linear recovery using a BEH 10 μm 300 Å-DEAP SPE plate.

FIG. 3 is a calibration curve for a large 50mer oligonucleotide showing linear recovery up to 12 μg/mL using a BEH 10 μm 300 Å-DEAP SPE plate.

FIG. 4 is a comparison of the recoveries observed with an Oasis™-based SPE plate and a BEH 10 μm 130 Å-DEAP SPE plate when extracting GEM 91 out of a plasma matrix.

FIGS. 5A-5C show percent recovery data upon a first elution step for oligonucleotides having five lengths ranging from 15 mer to 35 mer using the following stationary phase particles: stationary phase particles having diethylaminopropyl surface groups (FIG. 5A), stationary phase particles having 4-pyridylethyl surface groups (FIG. 5B), and stationary phase particles having piperazine surface groups (FIG. 5C).

FIGS. 6A-6B show percent recovery data upon an additional elution step for oligonucleotides having five lengths ranging from 15 mer to 35 mer using the following stationary phase particles: stationary phase particles having diethylaminopropyl surface groups (FIG. 6A) and stationary phase particles having piperazine surface groups (FIG. 6B).

### DETAILED DESCRIPTION

According to various aspects of the present disclosure, anion exchange sorbents are provided that are used, for example, in kits and methods for solid phase extractions. The anion exchange sorbents comprise a bulk material and ionizable surface groups that have one or more pKa's in a range of about 8 or less to about 12 or more. For example, the ionizable surface groups may have one or more pKa's ranging anywhere from 8 to 8.5 to 9 to 9.5 to 10 to 10.5 to 11 to 11.5 to 12 (i.e., ranging between any two of the preceding values).

In some embodiments, the ionizable surface groups may range from 0.01 to 10.0 μmol/m², for example, ranging from 0.01 to 0.05 to 0.10 to 0.30 to 0.50 to 1.0 to 3.0 to 5.0 to 10 μmol/m².

In some embodiments, the ionizable surface groups comprise amine groups such as secondary amine groups and/or tertiary amine groups, including, for example, —NHR₁ groups, —NR₁R₂ groups, and heterocyclic ring systems that contain at least one nitrogen atom, where R₁ and R₂ are independently selected from C₁-C₁₈ alkyl, C₂-C₁₈ alkenyl, C₂-C₁₈ alkynyl, C₃-C₁₈ cycloalkyl, C₃-C₁₈ heterocycloalkyl, C₆-C₁₈ aryl, or C₅-C₁₈ heteroaryl, for example, selected from C₁-C₄ alkyl, C₂-C₄ alkenyl, C₂-C₄ alkynyl, C₃-C₈ cycloalkyl, C₃-C₈ heterocycloalkyl, C₆-C₁₂ aryl, or C₆-C₁₂ heteroaryl, among others. Heterocyclic ring systems that contain at least one nitrogen atom may be selected, for example, from pyridyl, pyrimidinyl, pyridazinyl, pyrazinyl, piperidinyl, piperizinyl, hexahydropyrimidinyl, pyrrolyl, pyrazolyl, imidazolyl, pyrrolidinyl, pyrazolidinyl, imidazolidinyl, or triazinyl groups, among others. In embodiments where the ionizable surface groups comprise secondary amino groups, for example, —NHR₁ groups, the ionizable surface groups may comprise bulky groups, for example, bulky R₁ groups to suppress the nucleophilic activity of the ionizable surface groups.

In some embodiments, the ionizable surface groups may include a linking moiety that links the amine groups to the bulk material. Examples of linking moieties, include those comprising hydrocarbon groups, for example, C₁-C₁₈ alkylene, C₂-C₁₈ alkenylene, C₂-C₁₈ alkynylene or C₆-C₁₈ arylene groups, in some embodiments, C₁-C₄ alkylene, C₂-C₄ alkenylene, C₂-C₄ alkynylene or C₆-C₁₂ arylene groups. In some embodiments, the linking moieties may comprise non-reactive, non-hydrocarbon groups (e.g., an amide group, ester group, sulfo group, ether group, carbamate group, urea group, etc.) positioned between two hydrocarbon groups (e.g., independently selected from C₁-C₁₈ alkylene, C₂-C₁₈ alkenylene, C₂-C₁₈ alkynylene or C₆-C₁₈ arylene groups, in some embodiments, C₁-C₄ alkylene, C₂-C₄ alkenylene, C₂-C₄ alkynylene or C₆-C₁₂ arylene groups).

In certain embodiments, the ionizable surface groups may comprise aminoalkyl groups (e.g., amino-C₁-C₄-alkyl groups), alkylaminoalkyl groups (e.g., C₁-C₄-alkylamino-C₁-C₄-alkyl groups), or dialkylaminoalkyl groups (e.g., di-C₁-C₄-alkyl-amino-C₁-C₄-alkyl) groups). In certain embodiments, the ionizable surface groups may comprise methylaminomethyl, dimethylaminomethyl, ethylaminomethyl, diethylaminomethyl, methylaminoethyl, dimethylaminoethyl, ethylaminoethyl, diethylaminoethyl, methylaminopropyl, dimethylaminopropyl, ethylaminopropyl or diethylaminopropyl groups, among others.

In certain embodiments, the ionizable surface groups may be formed by reacting a bulk material with an organo-silane containing one or more amine groups. In certain embodiments, the ionizable surface groups may be formed by reacting a bulk material with an ionizable modifying reagents selected from one or more of the following, among others: 1-propanamine, 3-(dimethoxyphenylsilyl)-; 3-aminopropyldiisopropylethoxysilane; N-cyclohexylaminomethyltriethoxysilane; 2-(4-pyridylethyl)triethoxysilane; [3-(1-piperazinyl)propyl]triethoxysilane; N,N-diethylaminopropyl)trimethoxysilane; 3-aminopropyl)triethoxysilane; N-3-[(amino(polypropylenoxy)]aminopropyltrimethoxysilane; N,N′-bis(2-hydroxyethyl)-N,N′-bis(trimethoxysilylpropyl)ethylenediamine; N-(2-aminoethyl)-3-aminopropyltrimethoxysilane; N-cyclohexyl-3-aminopropyltrimethoxysilane; N-(2-aminoethyl)-3-aminopropylmethyldimethoxysilane 3-octadecanamine, 1-(trimethoxysilyl)-; 1-hexadecanamine, N,N-bis[3-(trimethoxysilyl)propyl]-; and 3,8-dioxa-4,7-disiladecan-5-amine, 4,4,7,7-tetraethoxy-N-hexadecyl-N-propyl.

The bulk material of the anion exchange sorbent may comprise, for example, a fully porous material or superficially porous material. The fully porous or superficially porous material of the anion exchange sorbent may be selected, for example, from (a) inorganic materials (e.g., silica, alumina, titania, zirconia), (b) inorganic-organic hybrid materials, (c) organic polymer materials, (d) a combination of (a) and (b), (e) a combination of (b) and (c), (f) a combination of (a) and (c), or (g) a combination of (a), (b) and (c), among other possibilities.

In various embodiments, the bulk material of the anion exchange sorbent may comprise a silica-based fully porous material or a silica-based superficially porous material. For example, the bulk material of the anion exchange sorbent may comprise a silica fully porous material or a silica superficially porous material in some embodiments.

In some embodiments, in addition to a fully porous material or a superficially porous material, the bulk materials used herein may further comprise a surrounding layer that comprises an inorganic-organic hybrid material.

In some embodiments, the fully porous material, the superficially porous material or the surrounding layer of the bulk material of the anion exchange sorbent may comprise a silica-based inorganic-organic hybrid material that includes silica regions in which the material comprises silicon atoms having four silicon-oxygen bonds and organosilica regions in which the material comprises silicon atoms having three, two or one silicon-oxygen bonds and one, two or three silicon-carbon bonds. In some cases the organosilica regions may comprise a substituted or unsubstituted alkylene, alkenylene, alkynylene or arylene moiety bridging two or more silicon atoms. For example the organosilica regions may comprise a substituted or unsubstituted C₁-C₁₈ alkylene, C₂-C₁₈ alkenylene, C₂-C₁₈ alkynylene or C₆-C₁₈ arylene moiety bridging two or more silicon atoms, in some embodiments, C₁-C₄ alkylene, C₂-C₄ alkenylene, C₂-C₄ alkynylene or C₆-C₁₂ arylene groups. In particular embodiments, the organosilica regions may comprise a substituted or unsubstituted C₁-C₆ alkylene moiety bridging two or more silicon atoms, including methylene, dimethylene or trimethylene moieties bridging two silicon atoms. In particular embodiments, the organosilica regions comprises may comprise ≡Si—(CH₂)ₙ—Si≡ moieties, where n is an integer equal to 1, 2, 3, or 4.

In some embodiments, the fully porous material, the superficially porous material or the surrounding layer of the bulk material of the anion exchange sorbent may comprise an inorganic-organic material of formula I:

(SiO₂)_{d}/[R²((R)ₚ(R¹)_{q}SiOₜ)ₘ] (I)

wherein,
R and R¹ are each independently C₁-C₁₈ alkoxy, C₁-C₁₈ alkyl, C₁-C₁₈ alkyl, C₂-C₁₈ alkenyl, C₂-C₁₈ alkynyl, C₃-C₁₈ cycloalkyl, C₁-C₁₈ heterocycloalkyl, C₅-C₁₈ aryl, C₅-C₁₈ aryloxy, or C₁-C₁₈ heteroaryl;
R² is C₁-C₁₈ alkyl, C₂-C₁₈ alkenyl, C₂-C₁₈ alkynyl, C₃-C₁₈ cycloalkyl, C₁-C₁₈ heterocycloalkyl, C₅-C₁₈ aryl, C₁-C₁₈ heteroaryl; or absent; wherein each R² is attached to two or more silicon atoms;
p and q are each independently 0.0 to 3.0;
t is 0.5, 1.0, or 1.5;
d is 0 to about 30;
m is an integer from 1-20; wherein R, R¹ and R² are optionally substituted;
provided that:
(1) when R² is absent, m=1 and

$$t = \frac{( 4 - ( p + q ) )}{2} ,$$

when 0<p+q≤3; and
(2) when R² is present, m=2-20 and

$$t = \frac{( 3 - ( p + q ) )}{2} ,$$

when p+q≤2.

In some embodiments, the fully porous material, the superficially porous material or the surrounding layer of the bulk material of the anion exchange sorbent may comprise an inorganic-organic material of formula II:

(SiO₂)_{d}/[(R)ₚ(R¹)_{q}SiOₜ] (II)

wherein,
R and R¹ are each independently C₁-C₁₈ alkoxy, C₁-C₁₈ alkyl, C₁-C₁₈ alkyl, C₂-C₁₈ alkenyl, C₂-C₁₈ alkynyl, C₃-C₁₈ cycloalkyl, C₁-C₁₈ heterocycloalkyl, C₅-C₁₈ aryl, C₅-C₁₈ aryloxy, or C₁-C₁₈ heteroaryl;
d is 0 to about 30;
p and q are each independently 0.0 to 3.0, provided that when p+q=1 then t=1.5; when p+q=2 then t=1; or when p+q=3 then t=0.5.

In some embodiments, the fully porous material, the superficially porous material or the surrounding layer of the of the bulk material anion exchange sorbent may comprise an inorganic-organic material of formula III:

(SiO₂)_{d}/[R²((R¹)ᵣSiOₜ)ₘ] (III)

wherein,
R¹ is C₁-C₁₈ alkoxy, C₁-C₁₈ alkyl, C₁-C₁₈ alkyl, C₂-C₁₈ alkenyl, C₂-C₁₈ alkynyl, C₃-C₁₈ cycloalkyl, C₁-C₁₈ heterocycloalkyl, C₅-C₁₈ aryl, C₅-C₁₈ aryloxy, or C₁-C₁₈ heteroaryl; R² is C₁-C₁₈ alkyl, C₂-C₁₈ alkenyl, C₂-C₁₈ alkynyl, C₃-C₁₈ cycloalkyl, C₁-C₁₈ heterocycloalkyl, C₅-C₁₈ aryl, C₁-C₁₈ heteroaryl; or absent; wherein each R² is attached to two or more silicon atoms;
d is 0 to about 30;
r is 0, 1 or 2, provided that when r=0 then t=1.5; or when r=1 then t=1; or when r=2 then t=0.5; and
m is an integer from 1-20.

In some embodiments, the fully porous material, the superficially porous material or the surrounding layer of the of the bulk material anion exchange sorbent may comprise an inorganic-organic material of formula IV:

(A)x(B)y(C)z (IV),

wherein the order of repeat units A, B, and C may be random, block, or a combination of random and block;
A is an organic repeat unit which is covalently bonded to one or more repeat units A or B via an organic bond;
B is an organosiloxane repeat unit which is bonded to one or more repeat units B or C via an inorganic siloxane bond and which may be further bonded to one or more repeat units A or B via an organic bond;
C is an inorganic repeat unit which is bonded to one or more repeat units B or C via an inorganic bond; and
x and y are positive numbers and z is a non-negative number, wherein x+y+z=1. In certain embodiments, when z=0, then 0.002≤x/y≤210, and when z≠0, then 0.0003≤y/z≤500 and 0.002≤x/(y+z)≤210.

In some embodiments, the fully porous material, the superficially porous material or the surrounding layer of the bulk material of the anion exchange sorbent may comprise an inorganic-organic material of formula V:

(A)x(B)y(B*)y*(C)z (V),

wherein the order of repeat units A, B, B*, and C may be random, block, or a combination of random and block;
A is an organic repeat unit which is covalently bonded to one or more repeat units A or B via an organic bond;
B is an organosiloxane repeat units which is bonded to one or more repeat units B or B* or C via an inorganic siloxane bond and which may be further bonded to one or more repeat units A or B via an organic bond;
B* is an organosiloxane repeat unit which is bonded to one or more repeat units B or B* or C via an inorganic siloxane bond, wherein B* is an organosiloxane repeat unit that does not have reactive (i.e., polymerizable) organic components and may further have a protected functional group that may be deprotected after polymerization;
C is an inorganic repeat unit which is bonded to one or more repeat units B or B* or C via an inorganic bond; and
x, y, and y* are positive numbers and z is a non-negative number, wherein x+y+z=1. In certain embodiments, when z=0, then 0.002≤x/(y+y*)≤210, and when z≠0, then 0.0003≤(y+y*)/z≤500 and 0.002≤x/(y+y*+z)≤210.

In embodiments where the fully porous material, the superficially porous material and/or the surrounding layer of the bulk material of the anion exchange sorbent comprise(s) an organic-inorganic hybrid material, the overall hybrid content of the bulk material of the anion exchange sorbent may range from 0.1 or less to 100 mol % hybrid, for example ranging from 0.1 to 0.25 to 0.5 to 1 to 2.5 to 10 to 25 to 50 to 75 to 100 mol % hybrid.

In various embodiments, the fully porous material, the superficially porous material and/or the surrounding layer of the bulk material of the anion exchange sorbent may be formed by hydrolytically condensing one or more silane compounds, which typically include (a) one or more silane compounds of the formula SiZ₁Z₂Z₃Z₄, where Z₁, Z₂, Z₃ and Z₄ are independently selected from Cl, Br, I, C₁-C₄ alkoxy, C₁-C₄ alkylamino, and C₁-C₄ alkyl, although at most three of Z₁, Z₂, Z₃ and Z₄ can be C₁-C₄ alkyl, for example, tetraalkoxysilanes, including tetra-C₁-C₄-alkoxysilanes such as tetramethoxysilane or tetraethoxysilane, alkyl-trialkoxysilanes, for example, C₁-C₄-alkyl-tri-C₁-C₄-alkoxysilanes, such as methyl triethoxysilane, methyl trimethoxysilane, or ethyl triethoxysilane, and dialkyl-dialkoxysilanes, for example, C₁-C₄-dialkyl-di-C₁-C₄-alkoxysilanes, such as dimethyl diethoxysilane, dimethyl dimethoxysilane, or diethyl diethoxysilane, among many other possibilities and/or (b) one or more compounds of the formula Si Z₁Z₂Z₃—R—SiZ₄Z₅Z₆, where Z₁, Z₂ and Z₃ are independently selected from Cl, Br, I, C₁-C₄ alkoxy, C₁-C₄ alkylamino, and C₁-C₄ alkyl, although at most two of Z₁, Z₂ and Z₃ can be C₁-C₄ alkyl, where Z₄, Z₅ and Z₆ are independently selected from Cl, Br, I, C₁-C₄ alkoxy, C₁-C₄ alkylamino, and C₁-C₄ alkyl, although at most two of Z₄, Z₅ and Z₆ can be C₁-C₄ alkyl, where R is an organic radical, for example, selected from C₁-C₁₈ alkylene, C₂-C₁₈ alkenylene, C₂-C₁₈ alkynylene or C₆-C₁₈ arylene groups. Examples include bis(trialkoxysilyl)alkanes, for instance, bis(tri-C₁-C₄-alkoxysilyl)C₁-C₄-alkanes such as bis(trimethoxysilyl)methane, bis(trimethoxysilyl)ethane, bis(triethoxysilyl)methane, and bis(triethoxysilyl)ethane, among many other possibilities.

In some embodiments, the fully porous material, the superficially porous material and/or the surrounding layer of the bulk material of the anion exchange sorbent may be formed by hydrolytically condensing one or more alkoxysilane compounds. Examples of alkoxysilane compounds include, for instance, tetraalkoxysilanes (e.g., tetramethoxysilane (TMOS), tetraethoxysilane (TEOS), etc.), alkylalkoxysilanes such as alkyltrialkoxysilanes (e.g., methyl trimethoxysilane, methyl triethoxysilane (MTOS), ethyl triethoxysilane, etc.) and bis(trialkoxysilyl)alkanes (e.g., bis(trimethoxysilyl)methane, bis(trimethoxysilyl)ethane, bis(triethoxysilyl)methane, bis(triethoxysilyl)ethane (BTE), etc.), as well as combinations of the foregoing. In certain of these embodiments, inorganic-organic hybrid silica-based materials may be prepared from two alkoxysilane compounds, for example, a tetraalkoxysilane such as TMOS or TEOS and an alkylalkoxysilane such as MTOS or a bis(trialkoxysilyl)alkane such as BTEE. When BTEE is employed as a monomer, the resulting materials are organic-inorganic hybrid materials, which are sometimes referred to as ethylene bridged hybrid (BEH) materials and can offer various advantages over conventional silica-based materials, including chemical and mechanical stability. One particular BEH material can be formed from hydrolytic condensation of TEOS and BTEE.

Further inorganic-organic hybrid materials are described in U.S. Pat. No. 6,686,035 B2, which is hereby incorporated by reference.

In various embodiments where the bulk material comprises surface silanol groups the concentration of surface silanol groups may be reduced by reaction with one or more suitable reactive organosilane compounds, for example, one or more silane compounds of the formula SiZ₇Z₈Z₉Z₁₀, where Z₇, Z₈, Z₉ and Z₁₀ are independently selected from Cl, Br, I, C₁-C₁₈ alkyl, C₂-C₁₈ alkenyl, C₂-C₁₈ alkynyl or C₆-C₁₈ aryl, wherein at least one and at most three of Z₇, Z₈, Z₉ and Z₁₀ is C₁-C₁₈ alkyl, C₂-C₁₈ alkenyl, C₂-C₁₈ alkynyl or C₆-C₁₈ aryl. In some embodiments, at least one and at most three of Z₇, Z₈, Z₉ and Z₁₀ is C₁-C₄ alkyl. In certain embodiments, silanol groups at a surface of the silica-based sorbents may be reduced in concentration by reaction with a haloalkylsilane compound selected from a chlorotrialkylsilane, a dichlorodialkylsilane or a trichloroalkylsilane, such as chlorotrimethylsilane, chlorotriethylsilane, dimethyldiclorosilane, diethyldiclorosilane, methyltrichlorosilane or ethyltrichlorosilane. In some embodiments, the reactive organosilane compounds provided in an amount sufficient to form organosilane surface groups in an amount ranging from 0.1 to 3.5 μmol/m².

As previously indicated, in various embodiments, the bulk material of the anion exchange sorbent may comprise an organic polymer material. For example, the fully porous material or the superficially porous material of the anion exchange sorbent may comprise an organic copolymer that comprises at least one hydrophobic organic monomer and at least one hydrophilic organic monomer.

In certain embodiments, the hydrophilic organic monomer may be selected from organic monomers having an amide group, organic monomers having an ester group, organic monomers having a carbonate group, organic monomers having a carbamate group, organic monomers having a urea group, organic monomers having a hydroxyl group, and organic monomers having nitrogen-containing heterocyclic group, among other possibilities. Specific examples of hydrophilic organic monomers include, for example, 2-vinylpyridine, 3-vinylpyridine, 4-vinylpyridine, N-vinylpyrrolidone, N-vinyl-piperidone, N-vinyl caprolactam, lower alkyl acrylates (e.g., methyl acrylate, ethyl acrylate, etc.), lower alkyl methacrylates (e.g., methyl methacrylate, ethyl methacrylate, etc.), vinyl acetate, acrylamide or methacrylamide, hydroxypolyethoxy allyl ether, ethoxy ethyl methacrylate, ethylene glycol dimethacrylate, or diallyl maleate. In particular embodiments, the hydrophilic organic monomer may be a monomer having the following formula,

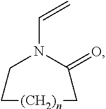

where n ranges from 1-3 (i.e., N-vinyl pyrrolidone, N-vinyl-2-piperidinone or N-vinyl caprolactam).

In certain embodiments, the hydrophobic organic monomer of the organic copolymer may comprise a C₂-C₁₈ olefin monomer and/or a monomer comprising a C₆-C₁₈ monocyclic or multicyclic carbocyclic group (e.g., a phenyl group, a phenylene group, naphthalene group, etc.). Specific examples of hydrophobic organic monomers include, for example, monofunctional and multifunctional aromatic monomers such as styrene and divinylbenzene, monofunctional and multifunctional olefin monomers such as ethylene, propylene or butylene, polycarbonate monomers, ethylene terephthalate, monofunctional and multifunctional fluorinated monomers such as fluoroethylene, 1,1-difluoroethylene), tetrafluoroethylene, chlorotrifluoroethylene, hexafluoropropylene, perfluoropropylvinylether, or perfluoromethylvinylether, monofunctional or multifunctional acrylate monomers having a higher alkyl or carbocyclic group, for example, monofunctional or multifunctional acrylate monomers having a C₆-C₁₈ alkyl, alkenyl or alkynyl group or a C₆-C₁₈ saturated, unsaturated or aromatic carbocyclic group, monofunctional or multifunctional methacrylate monomers having a higher alkyl or carbocyclic group, for example, monofunctional or multifunctional methacrylate monomers having a C₆-C₁₈ alkyl, alkenyl or alkynyl group or a C₆-C₁₈ saturated, unsaturated or aromatic carbocyclic group, among others. In certain embodiments, DVB 80 may be employed, which is an organic monomer mixture that comprises divinylbenzene (80%) as well as a mixture of ethyl-styrene isomers, diethylbenzene, and can include other isomers as well.

In certain embodiments, the organic copolymer may comprise a multifunctional hydrophobic organic monomer such as divinylbenzene and/or a multifunctional hydrophilic organic monomer, such as ethylene glycol dimethacrylate, methylene bisacrylamide or allyl methacrylate, in order to provide crosslinks in the organic copolymer.

In certain embodiments, the organic copolymer may comprise n-vinyl pyrrolidone or n-vinyl caprolactam as a hydrophilic organic monomer and divinylbenzene as a hydrophobic organic monomer.

In some embodiments, amine-containing ionizable surface groups such as those described above may be attached to the organic copolymer of the bulk material using suitable linking chemistry. As a specific example, after formation of a copolymer that comprises divinylbenzene, the divinylbenzene monomer within the copolymer can be chloromethylated, followed by amination along the lines described, for example, in U.S. Pat. No. 7,442,299, which is hereby incorporated by reference.

In various embodiments, in addition to a bulk material and ionizable surface groups, the anion exchange sorbents of the present disclosure may further comprise hydrophobic surface groups, for example, surface groups comprising hydrocarbon or fluorocarbon groups, typically alkyl groups, aromatic groups, or alkyl-aromatic groups, which may contain from 6 to 30 carbon atoms, and which are optionally substituted with one or more fluorine atoms.

In various embodiments, the porous anion exchange sorbents described herein may be in monolithic form.

In various embodiments, the porous anion exchange sorbents described herein may be in particulate form. For example, the porous anion exchange sorbents may be in the form of particles, typically spherical particles, having a diameter ranging from 1 to 100 m, for example, ranging 1 to 2 to 5 to 10 to 25 to 50 to 100 m, in some embodiments.

In various embodiments, the porous anion exchange sorbents described herein may have a pore size (average pore diameter) ranging from 75 to 2000 Angstroms, for example, ranging from 75 to 100 to 200 to 500 to 1000 to 2000 Angstroms as measured by conventional porosimetry methods. For sub-500 Angstrom pores, the average pore diameter (APD) can be measured using the multipoint N₂ sorption method (Micromeritics ASAP 2400; Micromeritics Instruments Inc., Norcross, Ga.), with APD being calculated from the desorption leg of the isotherm using the BJH method as is known in the art. Hg porosimetry may be used for pores that are 500 Angstrom or greater as is known in the art.

In some aspects of the present disclosure, anion exchange sorbents such as those described herein may be provided in conjunction with a suitable housing (referred to herein as a “sorbent housing”). The sorbent and the sorbent housing may be supplied independently, or the sorbent may be pre-packaged in the sorbent housing, for example, in a packed bed. Sorbent housings for use in accordance with the present disclosure commonly include a chamber for accepting and holding sorbent. In various embodiments, the sorbent housings may be provided with an inlet and an outlet.

Suitable construction materials for the sorbent housings include inorganic materials, for instance, metals such as stainless steel and ceramics such as glass, as well as synthetic polymeric materials such as polyethylene, polypropylene, polyether ether ketone (PEEK), and polytetrafluoroethylene, among others.

In certain embodiments, the sorbent housings may include one or more filters which act to hold the sorbent in a sorbent housing. Exemplary filters may be, for example, in a form of membrane, screen, frit or spherical porous filter.

In certain embodiments, a solution received in the sorbent housing may flow into the sorbent spontaneously, for example, by capillary action. In certain embodiments, the flow may be generated through the sorbent by external forces, such as gravity or centrifugation, or by applying a vacuum to an outlet of the sorbent housing or positive pressure to an inlet of the sorbent housing.

Specific examples of sorbent housings for use in the present disclosure include, for example, a syringe, a single-use injection cartridge, a multiple-use cartridge applicable for on-line SPE at pressures up to HPLC pressures (˜5000 psi) or higher pressures compatible with UHPLC (˜20000 psi), a column, a multi-well device such as a 4 to 8-well rack, a 4 to 8-well strip, a 48 to 96-well plate, or a 96 to 384-well micro-elution plate, micro-elution tip devices, including a 4 to 8-tip micro-elution strip, a 96 to 384-micro-elution tip array, a single micro-elution pipet tip, a thin layer plate, a microtiter plate, a spin tube or a spin container.

In various aspects of the present disclosure, anion exchange sorbents comprising ionizable surface groups having a pKa in a range of about 8 to about 12, more typically about 9 to about 12, even more typically 10 to 12, such as those described above, among other, are used in solid phase extraction procedures.

In some embodiments, the present disclosure provides methods of performing solid phase extraction that comprise (a) loading a sample comprising at least one target oligonucleotide and one or more matrix components (e.g., matrix components including proteins, lipids or both) onto a porous anion exchange sorbent comprising ionizable surface groups having a pKa in a range of about 8 to about 12, such as any of those described hereinabove, among others, whereby the at least one target oligonucleotide is retained by the sorbent, (b) flowing at least one washing solution though the sorbent, whereby the at least one washing solution removes the matrix components from the sorbent while leaving the at least one target oligonucleotide retained on the sorbent, and (c) flowing at least one elution solution though the sorbent, whereby the at least one elution solution releases the at least one target oligonucleotide retained on the sorbent.

As used herein the term “oligonucleotide” refers to a polymer sequence of two more nucleotides, including RNA, DNA, their analogs, including those having base modifications, sugar modifications or linkers used to modify the bioavailability, examples of which modifications include 2′-O-methoxyethyl, 2′-fluoro, phosphorothioate and or GalNAc modifications. Examples of oligonucleotides include antisense oligonucleotides (ASOs), small interfering RNAs (siRNAs), small hairpin RNAs (shRNAs), micro RNAs (miRNAs), messenger RNAs (mRNAs), and plasmids.

In one illustrative embodiment, in step (a), components such as salts, sugars and large proteins are able to flow through the sorbent, while the target oligonucleotide(s) of interest, smaller proteins and lipids are bound to the sorbent via a mixed-mode, weak anionic interaction. In step (b), the smaller proteins and lipids are washed from the sorbent, while the target oligonucleotide(s) of interest remain bound to the sorbent. In step (c), the now-purified target oligonucleotide(s) of interest are recovered from sorbent.

In various embodiments, the pore size of the porous anion exchange sorbent that is employed in the solid phase extraction methods will have a pore size that varies based on the length of the at least one target oligonucleotide.

For example, in embodiments where the one or more target oligonucleotide has/have a size ranging from 3 to 50 mer, a pore size ranging from 75 to 200 Angstroms may be selected. In embodiments where the one or more target oligonucleotide has/have a size ranging from 25 to 200 mer, a pore size ranging from 200 to 500 Angstroms may be selected. In embodiments where the one or more target oligonucleotide has/have a size ranging from 100 to 7000 mer, a pore size ranging from 500 to 2000 Angstroms may be selected

In some embodiments, the at least one washing solution used in the solid phase extraction may comprise an organic solvent, typically, 20 vol % to 100 vol %, for example, ranging from 20 vol % to 40 vol % to 60 vol % to 80 vol % to 90 vol % to 95 vol % to 98 vol % to 99 vol % to 100 vol % of an organic solvent such as methanol, acetonitrile or other common solvents used in reversed phase liquid chromatography, a salt such as up to 250 mM ammonium acetate, ammonium formate, or sodium chloride or other eluent solutions commonly used in ion exchange liquid chromatography, with pH controlled with ammonium acetate/formate or phosphate buffers or (semi)volatile buffers used in chromatography (e.g., morpholino buffers, ammonium acetate, triethylammonium acetate, etc.). In this regard, by using volatile buffers in the wash, the final eluent (extract) will contain as little non-volatile salt as possible. In some embodiments, the washing solution may have a pH ranging from 4 or less to 10 or more, for example, the washing solution may have a pH ranging anywhere from 4 to 5 to 6 to 7 to 8 to 9 to 10. The pH of the wash solution can be optimized for the particular porous anion exchange sorbent that is selected.

In some embodiments, the at least one elution solution used in the solid phase extraction may have a pH of at least 10, for example, ranging from 10 to 13, more typically, ranging from 10 to 12.

In some embodiments, the at least one elution solution used in the solid phase extraction may comprise a polyphosphonic acid. The polyphosphonic acid may be, for example, is a biphosphonic acid or a triphosphonic acid. The polyphosphonic acid may be selected, for example, from etidronic acid, clodronic acid, pamidronic acid, alendronic acid, neridronic acid, olpadronic acid, nitrilotri(methylphosphonic acid) or ethane-1,1,2-triphosphonic acid. In some embodiments, the at least one elution solution may comprise a polyphosphonic acid in a concentration ranging from about 0.01 M to about 0.1 M, for example, ranging from 0.01 M to 0.02 M to 0.05 M to 0.10 M to 0.20 M to 0.5 M to 1 M.

In some embodiments, the at least one elution solution used in the solid phase extraction may comprise one or more bases. The one or more bases may be selected from an organic amine, ammonium bicarbonate, ammonium hydroxide, or ammonium acetate. Organic amines include alkyl amines, for example, trimethyl amine, triethyl amine, or diisopropyl ethyl amine, among others.

In some embodiments, the at least one elution solution used in the solid phase extraction may comprise one or more organic solvents. The one or more organic solvents may be selected, for example, from methanol, ethanol, hexafluoroisopropanol (HFIP) and/or tetrahydrofuran, among others.

In particular embodiments, the at least one elution solution used in the solid phase extraction may comprise triethylamine (TEA), methanol and water, or the one or more elution solutions may comprise TEA, methanol, HFIP and water.

In some embodiments, the sample upon which the solid phase extraction is performed may be selected, for example, from biological fluids selected from whole blood samples, blood plasma samples, serum samples, oral fluids, cerebrospinal fluids, fecal samples, nasal samples, and urine, biological tissues such as liver, kidney and brain tissue, tissue homogenates, cells, or cell culture supernatants, among numerous other possibilities.

In some embodiments, the sample upon which the solid phase extraction is performed may be treated before loading the sample onto the porous anion exchange sorbent. For example, the sample may be treated with a denaturing agent. Suitable denaturing agents may be selected, for example, from proteases such as proteinase K, mass-spectroscopy-compatible surfactants, organic solvents, urea, guanidine, or a substituted guanidine.

In some embodiments, the substituted guanidine is selected from tetramethylguanidine, tertbutyl tetramethylguanidine, triazabicyclodecene, or combinations thereof. In some embodiments, the substituted guanidine comprises at least one from the group of tetramethylguanidine, tertbutyl tetramethylguanidine, triazabicyclodecene, or combinations thereof. In some embodiments, the substituted guanidine of tetramethylguanidine is 1,1,3,3-tetramethylguanidine with the chemical structure of

In some embodiments, the substituted guanidine of tertbutyl tetramethylguanidine is 2-tert-butyl-1,1,3,3-tetramethylguanidine with the chemical structure of

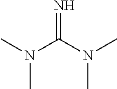

In some embodiments, the substituted guanidine of triazabicyclodecene is 1,5,7-triazabicyclo[4.4.0]dec-5-ene with the chemical structure of

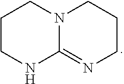

In some embodiments, the substituted guanidine is a guanidinium cation. In some embodiments, the substituted guanidine has a pKa value greater than about 10. In some embodiments, the concentration of the substituted guanidine is less than 250 mM.

In further aspects of the present disclosure, kits useful in performing solid phase extraction procedures may be provided. In various embodiments, the present disclosure provides kits that comprise a porous anion exchange sorbent comprising ionizable surface groups having a pKa in a range of about 8 to about 12, such as any of those described hereinabove, among others, a housing for the sorbent, such as any of those described hereinabove, among others, and one or more kit components selected from the following: (a) a denaturant solution, such as any of those described hereinabove, among others, (b) an elution solution, such as any of those described hereinabove, among others, (c) a washing solution, such as any of those described hereinabove, among others, (d) a collection plate or collection vial, (e) a cap mat, (f) calibration and reference standards, (g) instructions for use, and (h) identification tagging for each component, which may include passive tags, such as RFID tags, for tracking the components.

### EXAMPLES

### Example 1

Fully-porous silica particles were surface modified with an organic/inorganic hybrid surrounding material by hydrolytic condensation of TEOS and BTEE as described in U.S. Patent Pub. Nos. 2019/0091657 and 2019/0091657 to yield porous silica particles having an organic/inorganic surface.

### Example 2

Particles from Example 1 were functionalized by refluxing in toluene and an organo-silane containing one or more amine groups (see Table 1) for 2 h under anhydrous conditions. The particles were then isolated and washed in toluene, acetone, and acetone/water mixtures. The particles were then dried at elevated temperature under vacuum. Such particles can also be functionalized in the same manner using the additional ligands of Example 3.

TABLE 1

| | | Base | | | | | |
|---|---|---|---|---|---|---|---|
| | | Particle | | | | | |
| Sam- | Base | Surface | | | Final | Final | Ligand |
| ple | Par- | Area | Surrounding | Silane | Carbon | Nitrogen | Conc. |
| ID | ticle | (m²/g) | Material | Type | (%) | (%) | (μmol/m²) |
| 2a | Silica | 124 | (O_{1.5}SiCH₂CH₂SiO_{1.5})(SiO₂)₄ | 3-(Diethylamino)propyltrimethoxysilane | 1.29 | 0.04 | 0.26 |
| 2b | Silica | 123 | (O_{1.5}SiCH₂CH₂SiO_{1.5})(SiO₂)₄ | 3-(Diethylamino)propyltrimethoxysilane | 2.43 | 0.23 | 1.46 |

### Example 3

Fully-porous Ethylene Bridged Hybrid (BEH) particles can be prepared following the method as described in U.S. Pat. No. 6,686,035 or are commercially available in columns from available from Waters Corporation, Milford, Mass., USA, in various particle and pore sizes, including 10 m particles having 130 Å pore size (BEH 130) and 300 Å pore size (BEH 300). BEH particles were functionalized by refluxing in toluene and an organo-silane containing one or more amine groups (see Table 2) for 2 h under anhydrous conditions. The particles were then isolated and washed in toluene, acetone, and acetone/water mixtures. The particles were then dried at elevated temperature under vacuum. In some cases, the particles may be further functionalized (i.e., after completing the original functionalization) with ethyltrichlorosilane by refluxing the particles in toluene, silane, and pyridine for 20 h under anhydrous conditions then isolating the particles and washing in toluene, acetone and acetone/water mixtures.

TABLE 2

| | Base | Base | | | | |
|---|---|---|---|---|---|---|
| Sam- | Par- | Particle | | Final | Final | Ligand |
| ple | ticle | Surface | Silane | Carbon | Nitrogen | Concentration |
| ID | Type | Area (m²/g) | Type | (%) | (%) | (μmol/m²) |
| 3a | BEH | 188 | 3-(Diethylamino)propyltrimethoxysilane | 6.94 | 0.11 | 0.42 |
| 3b | BEH | 188 | 2-(4-Pyridylethyl)triethoxysilane | 6.51 | 0.03 | 0.11 |
| 3c | BEH | 188 | [3-(1-Piperazinyl)propyl]triethoxysilane | 7.02 | 0.20 | 0.38 |

### Example 4

Fully-porous silica particles were functionalized with [3-(diethylamino)propyl]trimethoxysilane (see Table 3) by refluxing the particles in toluene and silane for 2 h under anhydrous conditions then isolating the particles and washing in toluene, acetone, and acetone/water mixtures. The particles were then dried at elevated temperature under vacuum. In some cases, the particles were then further functionalized with ethyltrichlorosilane (see Sample ID 4c) by refluxing the particles in toluene, silane, and pyridine for 20 h under anhydrous conditions then isolating the particles and washing in toluene, acetone and acetone/water mixtures. The particles were then dried at elevated temperature under vacuum. Such particles can also be functionalized in the same manner using the additional ligands of Example 3.

TABLE 3

| | | Base | | | | |
|---|---|---|---|---|---|---|
| Sam- | Base | Particle | | Final | Final | Ligand |
| ple | Par- | Surface | Silane | Carbon | Nitrogen | Concentration |
| ID | ticle | Area (m²/g) | Type | (%) | (%) | (μmol/m²) |
| 4a | Silica | 120 | 3-(Diethylamino)propyltrimethoxysilane | 0.41 | 0.04 | 0.24 |
| 4b | Silica | 122 | 3-(Diethylamino)propyltrimethoxysilane | 0.52 | 0.05 | 0.29 |
| 4c | 4b | 122 | Ethyltrichlorosilane | 1.08 | 0.05 | 1.18 |

### Example 5

The organic/inorganic surrounding material as described in Example 1 can be modified to include an organo-silane containing one or more amine groups (e.g., [3-(diethylamino)propyl]trimethoxysilane) as a third component in addition to the TEOS and BTEE to yield particles having a surface composition similar to the final particles described in Example 2.

### Example 7

In this example, BEH 130 and BEH 300 particles having 3-(diethylamino)propyl ligands prepared in accordance with Example 3, and particles of an Oasis™-based anion exchanger having diethylamino functional groups, were used to analyze samples containing GEM 91, a fully thioated 25mer (mw 7776.3) and a custom synthesized 50mer (mw 15,879.6) from Integrated DNA Technologies (Coralville, Iowa, USA). Solutions were prepared containing the oligonucleotide in water.

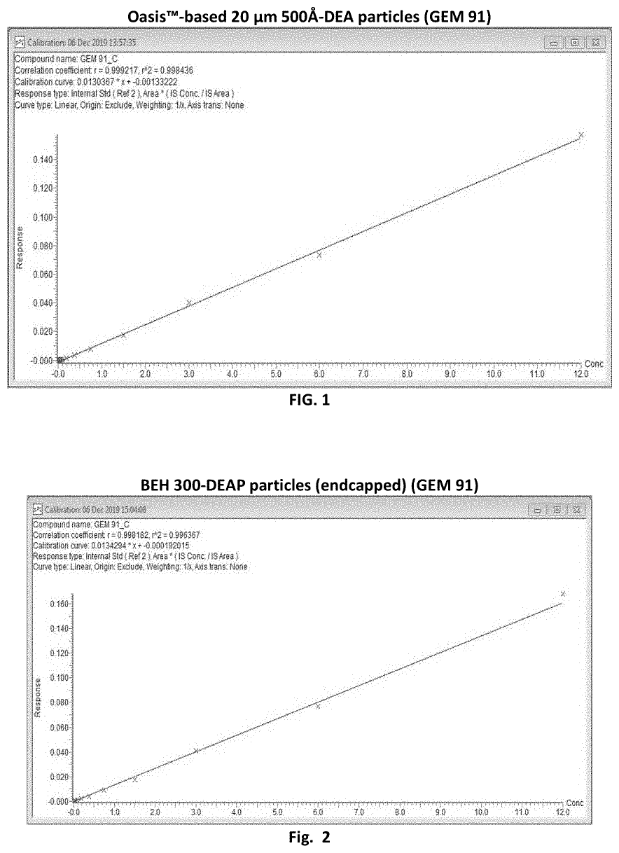

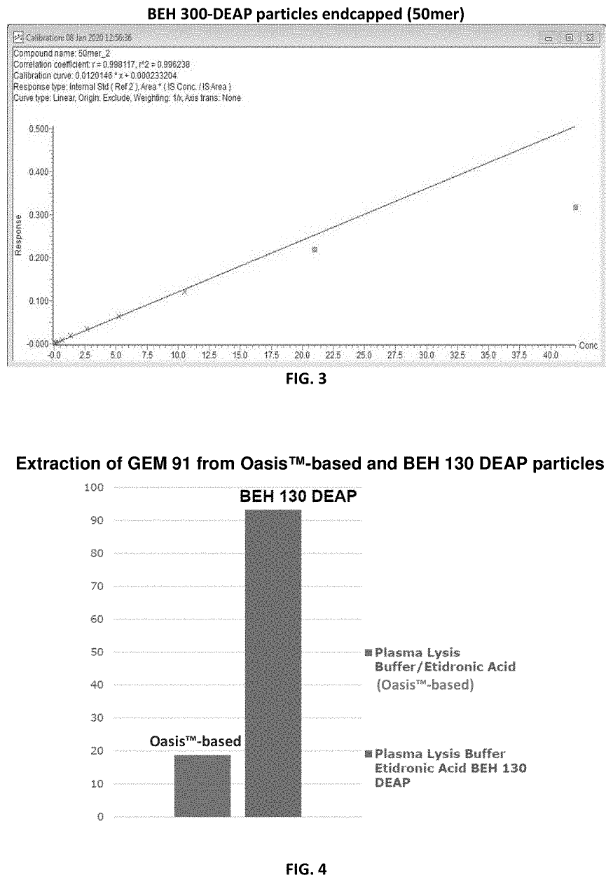

A 96 well plate loaded with approximately 2 mg of sorbent particles was used. The plate was conditioned with methanol and equilibrated with 50 mM ammonium acetate to pH 5.5. Sample was loaded onto the plate and washed twice with 50 mM ammonium acetate (pH 5.5) followed by two additional washes with 20:80 methanol:ammonium acetate (pH 5.5). The analytes were then eluted from the sorbent by using 2 elution washes of 20:80 methanol:50 mM TEA (pH 12) followed by 2 additional elution washes of 20:80 methanol:etidronic acid (pH 8). Samples were briefly evaporated under nitrogen at 70 C to evaporate the methanol before analysis. To evaluate recovery, standards were prepared in the elution solvents and added to the elution plate prior to evaporation. Samples were analyzed by LC/MS using negative ion MRM analysis. Quantitation was performed on the chromatographic peaks using Waters TargetLynx™ Application Manager software to integrate each peak relative to an internal standard and the peak area ratio was plotted against concentration. Results are shown in FIGS. 1-4.

The most effective ligand was found to be the 3-(diethylamino)propyl (DEAP—pKₐ˜11) on the BEH based material, which demonstrated almost complete recovery of the analyte from the plate.

Oasis™ 20 μm 500 Å-DEAP (GEM 91) demonstrated a recovery of ˜45%, with linearity seen from 188 ng/mL. BEH 300-DEAP endcapped (GEM 91), demonstrated >90% recovery and was linear from 23 ng/mL.

BEH 300-DEAP endcapped (50 mer) demonstrated 96% recovery, and was linear from 82 ng/ml.

### Example 7

In this example, stationary phase particles prepared in accordance with Example 3, specifically, stationary phase BEH 130 particles with diethylaminopropyl surface groups (having a pKa of ˜11), stationary phase BEH 130 particles with 4-pyridylethyl surface groups (having a pKa of ˜6.0), and stationary phase BEH 130 particles with piperazine surface groups (having a pKa of ˜9.8), were used to analyze oligonucleotides having lengths ranging of 15 mer, 20 mer, 25 mer, 30 mer and 35 mer were obtained from Integrated DNA Technologies. Solutions were prepared containing the oligonucleotides in water.

A 96 well plate loaded with approximately 2 mg of sorbent particles was used. The plate was conditioned with methanol and equilibrated with 50 mM ammonium acetate to pH 5.5. Each sample was loaded onto the plate and washed twice with 50 mM ammonium acetate (pH 5.5) followed by two additional washes with 20:80 methanol:ammonium acetate (pH 5.5). The analytes were then eluted from the sorbent by using 2 elution solutions of 50:50 MP B: 200 mM TEA, where MP B is formed from 50% MeOH, 7.5 mM TEA and 200 mM hexafluoroisopropanol (HFIP). Samples obtained from each of the first and second elution steps were briefly evaporated under nitrogen at 70° C. to evaporate the methanol and HFIP before analysis. To evaluate recovery, standards were prepared in the elution solvents and added to the elution plate prior to evaporation. Samples were analyzed by LC/MS using negative ion MRM analysis. Quantitation was performed on the chromatographic peaks using Waters TargetLynx™ Application Manager software to integrate each peak relative to an internal standard and the peak area ratio was plotted against concentration.

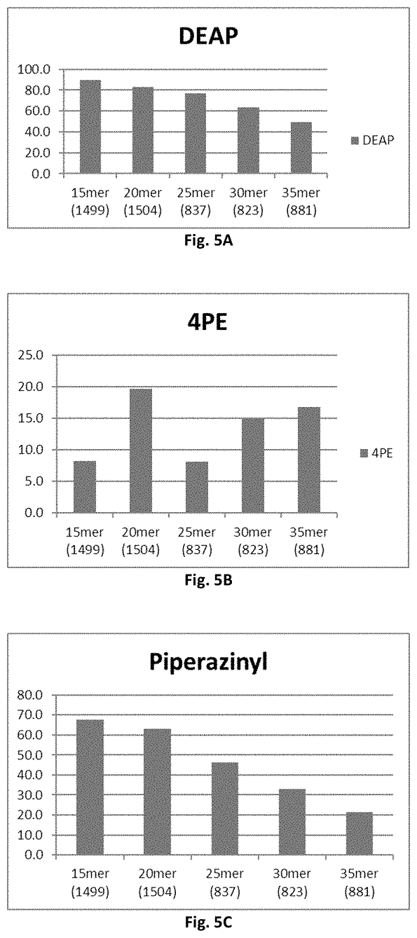

Results from the samples obtained from the first elution step are shown in FIGS. 5A-5C, which show that the percent recovery was highest for the stationary phase particles having diethylaminopropyl surface groups having a pKa of ˜11 (FIG. 5A) across all oligomers, the percent recovery was lowest for the stationary phase particles having 4-pyridylethyl surface groups (having a pKa of ˜6.0) (FIG. 5B), and the percent recovery was intermediate for the stationary phase particles having piperazine surface groups (having a pKa of ˜9.8) (FIG. 5C). Thus, percent recovery was seen to increase with increasing pKa.

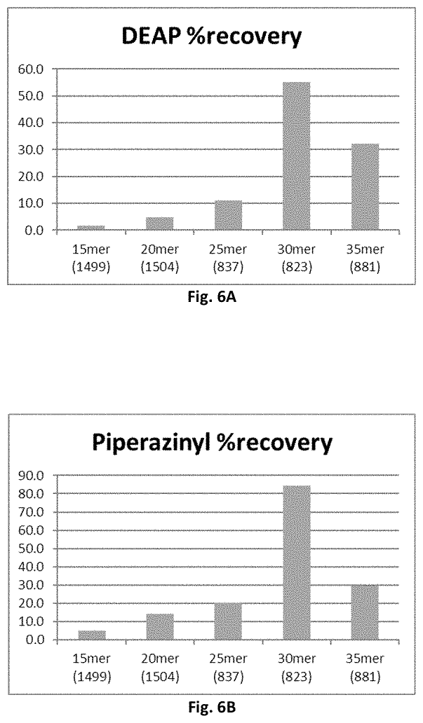

FIGS. 6A-6B show percent recovery data from the second elution step for the stationary phase particles having diethylaminopropyl surface groups (FIG. 6A) and the stationary phase particles having piperazine surface groups (FIG. 6B). These results show that the percent recovery was higher for the stationary phase particles having piperazine surface groups (FIG. 6B) relative to the stationary phase particles having diethylaminopropyl surface groups (FIG. 6A). This is believed to be due to the fact that the percent recovery for the particles having diethylaminopropyl surface groups was higher in the first elution step.

1. A method of performing solid phase extraction comprising:
loading a sample comprising one or more target oligonucleotides and one or more matrix components comprising proteins, lipids or both, onto a porous anion exchange sorbent comprising a bulk material and ionizable surface groups having a pKa in a range of about 8 to about 12, wherein target oligonucleotides are retained by the sorbent and matrix components are retained or unretained by the sorbent;
flowing one or more washing solutions through the sorbent, wherein the washing solutions remove any retained matrix components from the sorbent while leaving the target oligonucleotides retained on the sorbent; and
flowing one or more elution solutions though the sorbent, wherein the target oligonucleotides retained on the sorbent are released.

2. The method of claim 1, wherein the ionizable surface groups comprise amine-containing groups.

3. The method of claim 2, wherein the amine-containing groups are selected from —NHR₁ groups, —NR₁R₂ groups, and heterocyclic ring systems that contain at least one nitrogen atom, where R₁ and R₂ are independently selected from C₁-C₁₈ alkyl, C₂-C₁₈ alkenyl, C₂-C₁₈ alkynyl, C₃-C₁₈ cycloalkyl, C₃-C₁₈ heterocycloalkyl, C₆-C₁₈ aryl, or C₅-C₁₈ heteroaryl.

4. The method of claim 2, wherein the amine-containing groups comprise diethylaminopropyl (DEAP), diisopropylaminopropyl, ethylaminopropyl, dimethylaminopropyl, methylaminopropyl, aminopropyl, diethylaminoethyl, dimethylaminoethyl, dipropylaminoethyl, or diisopropylaminoethyl or diethylaminomethyl groups.

5. The method of claim 2, wherein the amine-containing groups are linked to the bulk material by linking moieties.

6. The method of claim 5, wherein the linking moieties comprise one or more of alkyl groups, amide groups, ester groups, sulfo groups, ether groups, carbamate groups and urea groups.

7. The method of claim 5, wherein the linking moieties comprise an amide group, ester group, sulfo group, ether group, carbamate group or urea group positioned between two C₁-C₆-alkyl groups.

8. The method of claim 1, wherein the bulk material comprises an inorganic material, a inorganic-organic hybrid material, an organic polymeric material, or a combination thereof.

9. The method of claim 1, wherein the bulk material comprises a silica-based material.

10. The method of claim 9, wherein the silica-based material comprises an inorganic-organic hybrid material that comprises silica regions in which the material comprises silicon atoms having four silicon-oxygen bonds and organosilica regions in which the material comprises silicon atoms having one or more silicon-oxygen bond and one or more silicon-carbon bonds.

11. The method of claim 10, wherein the organosilica regions comprise a substituted or unsubstituted alkylene, alkenylene, alkynylene or arylene moiety bridging two or more silicon atoms.

12. The method of claim 9, wherein silanol groups at a surface of the silica-based material are reduced in concentration by reaction with a C₁-C₁₈ alkyl silane compound.

13. The method of claim 1, wherein the porous anion exchange sorbent is in monolithic form or in particulate form.

14. The method of claim 1, wherein the one or more target oligonucleotides having a size ranging from a 3 mer to a 7000 mer.

15. The method of claim 1, wherein the porous anion exchange sorbent has a pore size ranging from 75 to 200 Angstroms and the sample contains one or more target oligonucleotides having a size ranging a 3 mer to a 50 mer, wherein the porous anion exchange sorbent has a pore size ranging from 200 to 500 Angstroms and the sample contains one or more target oligonucleotides having a size ranging a 25 mer to a 200 mer, and/or wherein the porous anion exchange sorbent has a pore size ranging from 500 to 2000 Angstroms and the sample contains one or more target oligonucleotides having a size ranging a 100 mer to a 7000 mer.

16. The method of claim 1, wherein the one or more washing solutions comprises an organic solvent and a volatile buffer.

17. The method of claim 1, wherein the one or more elution solutions have a pH ranging from 10 to 13.

18. The method of claim 1, wherein the one or more elution solutions comprise a polyphosphonic acid.

19. The method of claim 1, wherein the one or more elution solutions comprise one or more bases selected from an organic amine, ammonium bicarbonate, ammonium hydroxide, or ammonium acetate and one or more organic solvents selected from methanol, ethanol, or tetrahydrofuran.

20. The method of claim 19, wherein the one or more elution solutions comprise triethylamine (TEA) and methanol.

21. The method of claim 1, wherein the sample comprises biological fluids selected from whole blood samples, blood plasma samples, serum samples, oral fluids, cerebrospinal fluids, fecal samples, nasal samples, and urine, biological tissues such as liver, kidney and brain tissue, tissue homogenates, cells, or cell culture supernatants.

22. The method of claim 1, further comprising treating the sample with a denaturing agent before loading the sample onto the porous anion exchange sorbent.

23. The method of claim 22, wherein the denaturing agent is selected from a protease such as proteinase K, an MS compatible surfactant, an organic solvent, urea, guanidine, or a substituted guanidine.

24. A kit comprising a porous anion exchange sorbent comprising ionizable surface groups having a pKa in a range of about 8 to about 12, a housing for the sorbent, and one or more kit components selected from the following: a denaturant solution, an elution solution, or a washing solution.

25. The kit of claim 24, wherein the housing is selected from a multi-well strip, a multi-well plate, a single-use cartridge, or a multiple-use cartridge configured for on-line SPE.
